(19) United States
(12) Patent Application Publication
(10) Pub. No.: US 2008/0026102 A1
(43) Pub. Date: Jan. 31, 2008

(54) Liquid Concentrate Of Bacteria That Are Adapted And Fit For Alimentary Use

(75) Inventors: Luc Terragno, Paris (FR); Guillaume Catonnet, Massy (FR); Pascal Regulier, Guyancourt (FR); Christophe Daval, Choisy-Le-Roi (FR); Philippe Teissier, Massy (FR); Jean-Yves Barbeau, Igny (FR)

Correspondence Address: FOLEY AND LARDNER LLP;SUITE 500, 3000 K STREET NW, WASHINGTON, DC, 20007, US

(73) Assignee: COMPAGNIE GERVAIS DANONE, LEVALLOIS-PERRET FRANCE (FR)

(21) Appl. No.: 10/590,507

(22) PCT Filed: Feb. 28, 2005
(86) PCT No.: PCT/FR05/00478

§ 371 Date: Dec. 1, 2006

(30) Foreign Application Priority Data
Feb. 27, 2004 (FR) 0401997

(51) Int. Cl. A23L 1/30; A23C 9/12; B65D 85/50; C12N 1/20; A23C 9/127

(52) U.S. Cl. 426/61; 206/524.1; 435/252.1; 435/252.5; 435/252.9; 435/253.4

(57) ABSTRACT

The invention relates to a liquid concentrate of bacteria that are adapted and fit for alimentary use. In a preferred but not limited manner, the bacteria produced are lactic bacteria.

This invention relates to a liquid concentrate of bacteria that are adapted and viable for food use. Preferably, but in a non-limiting manner, the bacteria produced are lactic bacteria.

The ingestion of certain bacteria strains, in particular those that belong to the Lactobacillus and Bifid bacterium genera, are particularly beneficial for health, in particular by promoting proper functioning of the intestinal flora. Indeed, these bacteria produce bacteriocines and lactic acid, which increase the digestibility of foods, promote intestinal peristalsis, and accelerate the evacuation of stools. Moreover, these bacteria produce certain B-complex vitamins, and generally promote the absorption of vitamins and minerals, reduce blood cholesterol, strengthen the immune system and line the intestinal mucous membranes so as to protect them from invasion and the action of harmful microorganisms.

Therefore, for a number of years, agro-food industries have attempted to incorporate such bacteria in their final products, most generally yogurts.

Currently, these bacteria are used in frozen or lyophilized form. However, these production processes are traumatic for the bacteria, which lose some of their activity and sometimes their viability. This is detrimental for the manufacturers and for the consumers of these products because the bacteria must satisfy the requirements of quality and technological performance, if possible for a number of months. It would therefore be desirable to use bacteria produced by a method ensuring their viability and maximum activity. To this end, a method consists of producing the bacteria in liquid form. However, it has been demonstrated that this method also causes a high rate of death of the bacteria, after the bacteria are added to the final product.

In addition, to reduce the costs of storage of bacteria and to facilitate the addition of the bacteria to the final product, it would be desirable to concentrate the bacteria in liquid form. To do this, a person skilled in the art normally uses a centrifugation or filtration step. However, centrifugation is a traumatic process for the bacteria that can cause significant cell death in particular due to a strong shear force, and, moreover, this method is not very suitable for the centrifugation of small amounts such as those required in the production of bacteria intended to be added as a probiotic to food products. As regards a conventional filtration step, it also presents problems of bacteria death and clogging of the filters by the bacteria.

It would therefore be desirable to produce a desired amount of liquid concentrate of bacteria that have a maximum viability and activity after the concentration step and after being added to the final product.

Surprisingly and unexpectedly, the inventors showed that a step of adapting the bacteria made it possible to significantly increase the activity and the viability of the bacteria after they were added to the final product.

Moreover, the inventors showed that a tangential filtration step, under certain specific conditions (pressure, concentration, membrane porosity, etc.), made it possible to concentrate large amounts of bacteria culture, while preserving the viability and without clogging the filters.

Tangential filtration makes it possible to produce two currents according to the type and structure of the membrane: the permeate (the culture medium substantially free of bacteria) and the retentate (containing the bacteria, also called concentrate). In tangential filtration, the fluid circulates not perpendicularly but parallel to the surface of the membrane and thus ensures, by its flow speed, self-cleaning that prevents the accumulation of deposits that obstruct the filtration surface (also commonly called filter clogging).

An object of this invention is therefore a bacteria concentrate characterised in that the concentrate is liquid and in that the bacteria are adapted, viable and at a concentration between 5.10¹⁰ and 5.10¹¹ ufc/ml.

By bacteria, we are preferably referring, according to this invention, to lactic bacteria of the following genera: Lactobacillus spp., Bifidobacterium spp., Streptococcus spp., Lactococcus spp. and in particular Lactobacillus casei, Lactobacillus plantarum, Lactobacillus bulgaricus, Lactobacillus helveticus, Lactobacillus acidophilus, Bifidobacterium animalis, Bifidobacterium breve, Streptococcus thermophilus and Lactococcus lactis.

By adapted bacteria, we are referring, according to this invention, to bacteria that are more resistant to various stresses, in particular associated with various physicochemical stresses.

By adapted and viable bacteria, we are referring, according to this invention, to bacteria having a survival rate, after 28 days in a food product, in particular a dairy product or a drink, of above 60%, and advantageously above 80%.

The viability of bacteria is measured by counting techniques known to a person skilled in the art, such as, for example, mass count, surface count, Malassez cells, direct counting, turbidity, nephelometry, electronic counting, flow cytometry, fluorescence, impedimetry and image analysis.

According to this invention, the concentrate is characterised in that the adapted bacteria have at least one of the following characteristics when they are added to a food product:

i) a survival rate above 80% after 14 days in a food product at a temperature between 4° C. and 45° C., with said food product having a pH between 3 and 7, or

ii) a survival rate above 60% and advantageously above 80% after 28 days in a food product at a temperature between 4° C. and 45° C., with said food product having a pH between 3 and 7.

According to this invention, the concentrate is characterised in that the bacteria have both characteristics i) and ii).

According to this invention, the concentrate is characterised in that the food product is a dairy product and/or a drink.

According to this invention, the bacteria of the concentrate are viable for a period of between 4 and 6 weeks.

According to this invention, the concentrate is characterised in that it is capable of being obtained by the method including the successive steps of propagation of the bacteria in a culture medium, adaptation of the bacteria, washing of the culture medium containing the adapted bacteria by tangential microfiltration, and concentration of bacteria in the washed medium by tangential microfiltration.

According to this invention, the culture medium of the propagation step is a synthetic medium.

By synthetic medium, we are referring, according to this invention, to a medium to which components subjected to a strict quantitative and qualitative control are added.

According to this invention, the solution used in the washing step is suitable for the food use of the bacteria concentrate and has an osmotic pressure compatible with the viability of the bacteria.

The inventors showed that the step of adaptation of the bacteria makes it possible to reduce the mortality thereof, caused by the change in medium of the bacteria between their culture medium and the final food product to which they are added.

According to this invention, the adaptation of the bacteria is determined by the measurement of parameters of the bacteria culture medium and/or parameters of the bacteria. According to this invention, the parameters of the culture medium are preferably the pH, the osmotic pressure and/or the temperature.

Other parameters of the bacteria culture medium for determining the adaptation of bacteria are possible, such as, for example, the sugar concentration of the bacterial medium.

Preferably, if the parameter of the culture medium is the pH, the adaptation step is performed by reducing the pH by natural acidification.

To perform the step of adaptation of the bacteria to the pH by natural acidification, it is possible, for example, to measure the sugar concentration of the fermentation medium and, beyond a threshold concentration for each bacteria species, it is known that the pH is no longer regulated, and it becomes very easy to adapt the medium.

Thus, for example, if the sugar concentration of the fermentation medium of Lactobacillus casei is 9 g/L, the pH is no longer regulated and is equal to around 5. It then becomes easier for the adapted strain to be added to a new medium, and this allows for better viability of the bacteria in the final medium.

In addition, according to this invention, tangential filtration can be used for the bacteria adaptation step.

According to this invention, the tangential filtration membrane(s) have a porosity between 0.01 and 0.05 μm, and preferably between 0.1 and 0.3 μm.

These membranes are used for the washing and concentration steps of the method and possibly the bacteria adaptation step.

The filtration membranes are characterised by:
the porosity and thickness of the filtering layer, on which the permeate flow is dependent;
the diameter of the pores and their distribution, on which the separation efficacy is dependent;
the material used, on which the mechanical, chemical and thermal resistance and the ease of cleaning are dependent.

By filtration membrane, we are referring to organic or mineral membranes.

The organic membranes can consist, inter alia, of cellulose acetate, aromatic polyamides, polysulfone, cellulose esters, cellulose, cellulose nitrate, PVC or polypropylene.

The mineral membranes can consist, inter alia, of sintered ceramic, sintered metal, carbon or glass.

According to this invention, the parameter of the bacteria is the size thereof.

Preferably, if the adaptation is determined by the size of the bacteria, the distribution of the lengths of each bacteria of said concentrate is primarily between 0.1 and 10 micrometers, and advantageously between 0.5 and 5 micrometers.

The measurement of the size of the bacteria is performed using appropriate means.

Appropriate means can be, for example, a regular sample of bacteria followed by a measurement of the bacteria size by flow cytometry.

According to this invention, the pH of the concentrate is between 3 and 6.

According to this invention, the temperature of use of the concentrate is between 25 and 45° C. and preferably between 35 and 39° C.

By temperature of use, we are referring in this invention to the temperature of the concentrate when it is added to a food product.

According to this invention, the concentrate is packaged in flexible, hermetically sealed and sterile bags.

By flexible and hermetically sealed bags, we are referring in this invention preferably to food-safe plastic bags.

According to this invention, the concentrate, packaged in flexible, hermetically sealed bags, can be preserved at a temperature of between −50 and 4° C. after packaging.

Optionally, it is possible to add cryoprotective molecules, such as saccharose, for example, to the liquid concentrate of adapted and viable bacteria packaged in flexible and hermetically sealed bags and conserved at low temperatures.

According to this invention, the concentrate, packaged in flexible and hermetically sealed bags, preserved at a temperature of between −50 and 4° C., is reheated to a temperature of between 25 and 45° C., and advantageously between 35 and 39° C., by appropriate means before being used.

By appropriate means, we are referring in this invention, for example, to the use of a bain-marie at a non-lethal temperature for the bacteria, for example 37° C.

An object of this invention is also the use of the liquid concentrate of adapted and viable bacteria, according to this invention, as a food additive.

By food additive, we are referring in this invention to any chemical substance added to food during its preparation or for storage purposes so as to obtain a desired technical effect. In addition, according to this invention, the liquid concentrate of bacteria has a stable count, as the bacteria are viable and do not ferment in the final product to which they are added.

An object of this invention is also a food product to which a substance is added, characterised in that the food additive used is the liquid concentrate of adapted and viable bacteria according to this invention.

According to this invention, the food product is a dairy product and/or a drink.

By dairy product, we are referring in this invention, to, in addition to milk, milk-derived products such as cream, ice cream, butter, cheese, yogurt; secondary products, such as whey, casein as well as various prepared foods containing milk or milk constituents as the main ingredient.

By drink, we are referring in this invention to drinks such as, for example, fruit juices, mixtures of milk and fruit juices, plant-based juices such as, for example, soy milk, oat milk or rice milk, alcoholic drinks such as, for example, kefir, sodas, and spring or mineral water to which sugar or flavourings may or may not be added, for example.

An object of this invention is also a method for producing a food product to which a substance is added according to this invention, characterised in that the liquid concentrate of adapted and viable bacteria is added to the food product at the end of the production line and preferably before the packaging of the food product.

According to this invention, the method for producing a food product to which a substance is been added is characterised in that the liquid concentrate of adapted and viable bacteria is added to the food product in the line by pumping.

This invention can be better understood with the following description, which refers to examples of measuring the viability and adaptation of the bacteria of the liquid concentrate according to this invention.

It goes without saying, however, that these examples are provided only to illustrate the object of the invention, and can in no way constitute a limitation.

### LEGEND OF THE FIGURES

FIG. 1: representation of the viability of adapted and non-adapted Lactobacillus casei strains in a yogurt-type food product over a 28-day period;

FIG. 2a: histogram of size distribution of adapted and non-adapted Lactobacillus casei strains before being subjected to acid stress.

FIG. 2b: histogram of size distribution of adapted and non-adapted Lactobacillus casei strains after being subjected to acid stress.

FIG. 3: Curves showing the adaptation of Lactobacillus casei with respect to the temperature of the culture medium (37 and 39° C.). The relative permittivity ε_{R} (dimensionless amount equal to the permittivity ε, expressed in pF/cm, divided by the permittivity of the vacuum ε₀) is expressed according to the age of the bacteria (in hours)

FIG. 4: Curves showing the adaptation of Lactobacillus casei as a function of the osmotic pressure (glucose concentration of 20, 40, and 80 g/L, respectively in light grey, dark grey and black). The permittivity ε, expressed in pF/cm, is expressed as a function of the optical density (OD).

### EXAMPLES

### Example 1

### Consequences of the Adaptation of L. casei Strains on Their Viability

The consequences of a step of adaptation of Lactobacillus casei strains on their viability are to be evaluated.

To do this, a batch of Lactobacillus casei control bacteria is prepared and placed in culture in a MRS medium (special medium allowing for the growth of Lactobacilli, developed by Man, de Rogosa and Sharpe).

Simultaneously, a batch of Lactobacillus casei bacteria is prepared, which, after being placed in culture in a MRS medium, is adapted by a step of natural acidification.

To do this, after 17 hours of culture, a reduction in the pH is achieved by natural acidification over one hour to change it from pH 6.5 to pH 5.

Then, the two batches of bacteria are washed and concentrated in bacteria by tangential microfiltration.

These two bacteria concentrates are separately added to a yogurt mass at pH 5.5 and at a temperature of 10° C.

The amount of living bacteria at D+1 is measured in the two yogurt batches.

Then, every day, a sample is taken from the two batches of yogurt to which the control bacteria concentrate and the adapted bacteria concentrate, respectively, have been added, and the number of surviving strains is quantified with respect to the number of living strains at D+1. A mass count is used for this.

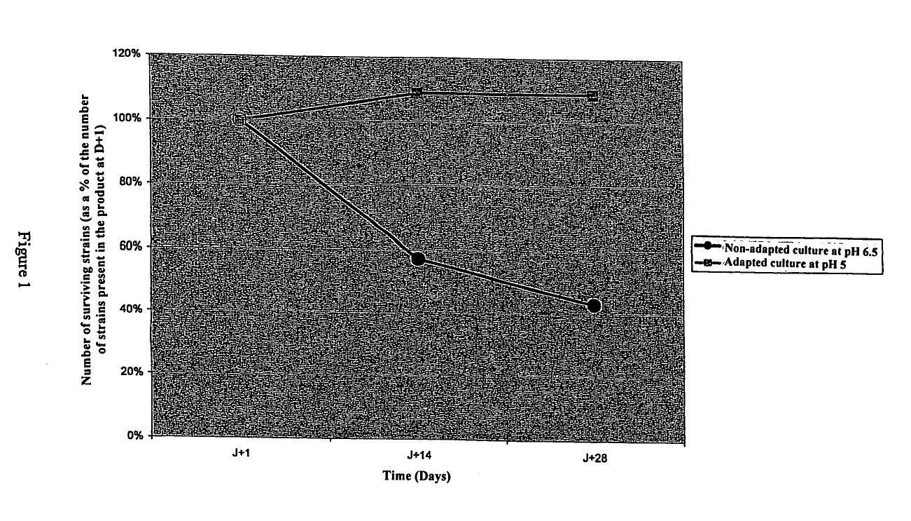

For each of the viability measurements during the period of preservation of the final product, the latter is well homogenized before the sample is collected. A sterile sample of 1 ml of product is collected. A serial dilution by factors of 10 is performed. The various dilutions of the product are placed in a Petri dish and a liquid agar medium (since previously heated at 50° C.) is poured over these fractions of the product. The medium to be poured will be selected according to the type of bacteria to be counted. The agar medium hardens. The Petri dishes are then placed in incubation for a few days (2 to 5 d) at 37° C. The results are shown in FIG. 1.

After 7 days, it is observed that the number of surviving bacteria in the control bacteria batch is 80% and that in the adapted bacteria batch is 105% (there was slight bacterial growth).

After 14 days, the number of surviving bacteria in the control bacteria batch is 58% and that in the adapted bacteria batch is 110% (there was slight bacterial growth).

After 28 days, the number of surviving bacteria in the control bacteria batch is 42% and that in the adapted bacteria batch is 110% (there was slight bacterial growth).

To conclude, the bacteria adaptation step causes a decrease in the mortality of the bacteria on the order of 60% with respect to a batch of non-adapted control bacteria, after 28 days in yogurt.

### Example 2

### Change in the Size of Adapted Bacteria and Non-Adapted Bacteria Subjected to Acid Stress

The change in size of adapted bacteria and non-adapted bacteria subjected to acid stress is to be monitored.

To do this, a batch of Lactobacillus casei control bacteria is prepared and placed in culture in an MRS medium (special medium allowing for the growth of Lactobacilli, developed by Man, de Rogosa and Sharpe).

Simultaneously, a batch of Lactobacillus casei bacteria is prepared, which, after being placed in culture in an MRS medium, is adapted by a step of natural acidification.

To do this, after 17 hours of culture, a reduction in the pH is achieved by natural acidification over one hour to change it from pH 6.5 to pH 5.

Then, the two batches of bacteria are washed and concentrated in bacteria by tangential microfiltration.

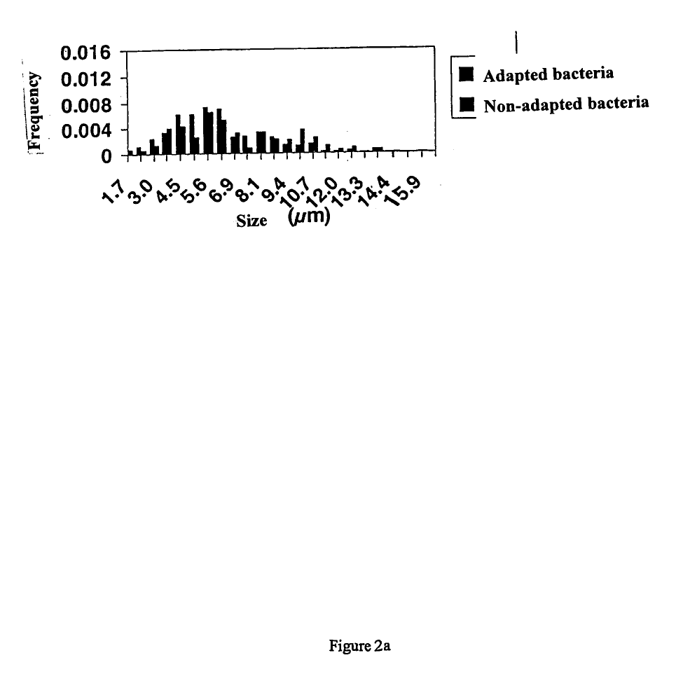

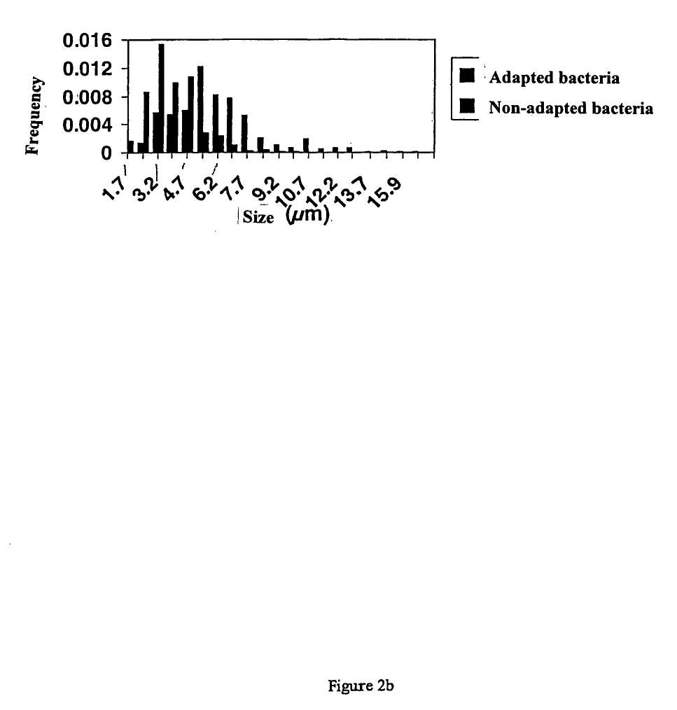

Then, a sample of the bacteria is collected and their size is measured by flow cytometry. A histogram of adapted and non-adapted (control batch) bacteria size distribution is thus established (FIG. 2a). It is observed that the bacteria size distribution of the two lots is very similar.

These two batches of bacteria are then subjected to acid stress by adding bacteria to a medium having a Ph of 3.

Then a sample of the bacteria is collected and their size is measured by flow cytometry. A histogram of adapted and non-adapted (control batch) bacteria size distribution after acid stress is thus established (FIG. 2b). It is observed that the bacteria size distribution of the two lots is very different. In the batch of adapted bacteria, the largest size frequency is 3.2 μm (frequency of 0.016). In the batch of non-adapted bacteria, the largest size frequency is 5.45 μm (frequency of 0.012).

To conclude, the bacteria adaptation step causes a decrease in the bacteria size on the order of 60% when they are subjected to acid stress, with respect to a batch of non-adapted control bacteria. It is therefore possible to show the adaptation of bacteria by measuring their size.

### Example 3

### Determination of the Adaptation of Bacteria by Measuring the Influence of the Parameter of “Temperature” of the Culture Medium

The adaptation of bacteria is to be determined by measuring the influence of the temperature parameter of the culture medium.

To do this, two batches of Lactobacillus casei bacteria are prepared from the same inoculum. These two batches are then placed in culture in two MRS media (special medium allowing for the growth of Lactobacilli, developed by Man, de Rogosa and Sharpe).

A biomass sensor enabling the relative permittivity ε_{R} to be measured is used. The probes that can be used for this purpose are known to a person skilled in the art (see in particular FR 2835921). The relative permittivity ε_{R} is a dimensionless amount equal to the permittivity ε (expressed in pF/cm) divided by the permittivity of the vacuum ε₀ (ε₀=8.854187×10⁻² pF/cm). The change in relative permittivity is measured over time. The relative permittivity will be dependent on the number of living cells and the size of these cells.

The two strictly identical culture media comprising the same number of bacteria are cultivated, one at 37° C. and the other at 39° C.

The number of strains increases over time. This is normal.

The inventors were able to verify that the total number of cells was no different between the two culture media. A conventional technique for measuring the optical density (absorption spectrometry) can be used to this end, or a Wedgewood optical probe can be used (system measuring the optical density of microbial suspensions in the near infrared). The absorbency of the medium measured by a spectrometer will be dependent on the total number of cells in the medium. Techniques for counting in Petri dishes can also be used.

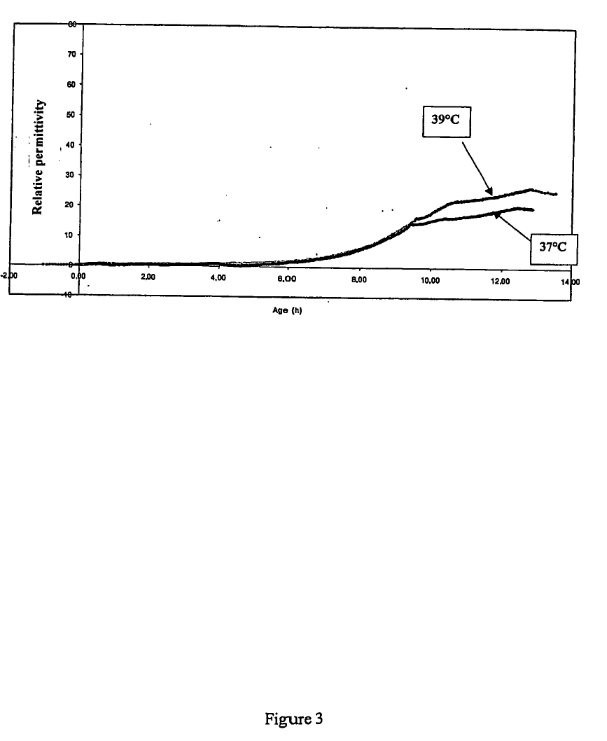

With the biomass sensor used, it was possible to demonstrate that the bacteria changed shape and size, and therefore adapted according to the temperature of the culture medium. FIG. 3 shows this observation.

### Example 4

### Determination of the Adaptation of Bacteria by Measuring the Influence of the Parameters of “Temperature” and “pH” of the Culture Medium

The adaptation of bacteria is to be determined by measuring the influence of two parameters of the culture medium, which are temperature and pH.

To do this, three batches of Lactobacillus casei bacteria are prepared from the same inoculum.

These three culture media are cultivated at three different temperatures (35° C., 37° C. and 39° C.) while subjecting the bacteria each time to acid stress by lowering the pH of the medium to a pH of 3.

The change in size of the cells will result in their adaptation to the conditions of the medium. This size will be measured by microscopy.

In Table 1, the results obtained clearly show that the bacteria adapt according to the temperature and pH parameters of the medium since these bacteria change size.

TABLE 1
Influence of the temperature and pH on the size
of the bacteria (expressed in μm)

| | Average size | Average size | Average size |
|---|---|---|---|
| | of bacteria | of bacteria | of bacteria |
| Temperature | during the | before change | after change |
| (° C.) | preculture | in pH | in pH |
| 35 | 3 | 3 | 2 |
| 37 | 4 | 5 | 4 |
| 39 | 3 | 7 | 5 |

### Example 5

### Determination of the Adaptation of Bacteria by Measuring the Influence of the Parameter of “Osmotic Pressure” of the Culture Medium

The adaptation of bacteria is to be determined by measuring the influence of the parameter of osmotic pressure of the culture medium.

To do this, three batches of Lactobacillus casei bacteria are prepared from the same inoculum. These batches are placed in culture in an MRS media (special medium allowing for the growth of Lactobacilli, developed by Man, de Rogosa and Sharpe).

The three culture media comprising the same number of bacteria respectively contain amounts of 20, 40, and 80 g of glucose per litre of culture medium. The higher the glucose concentration of the medium is, the higher the osmotic pressure of said medium is.

A sensor enabling the relative permittivity to be measured is used. The probes that can be used for this purpose are known to a person skilled in the art (FR 2835921). The change in this relative permittivity over time is measured. The permittivity ε (expressed in pF/cm) is calculated by multiplying the measured relative permittivity ε_{R} by the permittivity of the vacuum ε₀ (ε₀=8.854187×10⁻² pF/cm).

A conventional technique for measuring the optical density (absorption spectrometry), or a Wedgewood optical probe (system measuring the optical density of microbial suspensions in the near infrared), is used to measure the change in optical density of the medium over time. The optical density (OD) value obtained will be dependent on the total number of cells in the medium.

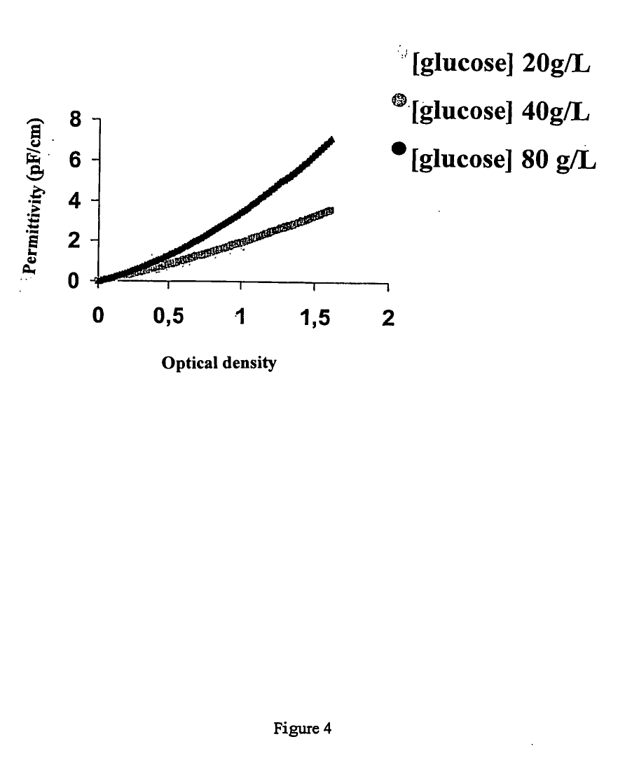

By expressing the permittivity as a function of the OD, the resulting curve (FIG. 4) makes it possible to determine the change in viability (expressed by the permittivity measurement) and the size of the cells as a function of the osmotic pressure of the medium. The results show that the bacteria change size. Indeed, if there is no change in size of the cells, the results observed in FIG. 4 would be straight lines. In this case, curves can be seen (nonlinear correlation).

The bacteria therefore adapt according to the osmotic pressure of the culture medium.

1. A bacteria concentrate in liquid form comprising adapted and viable bacteria at a concentration between 5.1010 and 5.1011 ufc/ml, said adapted and viable bacteria being more resistant to various physiochemical stresses.

2. The concentrate according to claim 1, wherein the bacteria are lactic bacteria, Lactobacillus spp., Bifid bacterium spp., Streptococcus spp. or Lactococcus spp. genera.

3. The concentrate according to claim 1, wherein the adapted bacteria have at least one of the following characteristics when they are added to a food product:
i) a survival rate above 80% after 14 days in a food product at a temperature between 4 C and 45 C, with said food product having a pH between 3 and 7, or
ii) a survival rate above 60% and advantageously above 80% after 28 days in a food product at a temperature between 4 C and 45 C, with said food product having a pH between 3 and 7.

4. The concentrate according to claim 1, wherein the bacteria have both characteristics i) and ii).

5. The concentrate according to claim 1, wherein the food product is a dairy product and/or a drink.

6. The concentrate according to claim 1, wherein the bacteria are viable for a period of between 4 and 6 weeks.

7. The concentrate according to claim 1, wherein it is capable of being obtained by the method including the successive steps of propagation of the bacteria in a culture medium, adaptation of the bacteria, washing of the culture medium containing the adapted bacteria by tangential microfiltration, and concentration of bacteria in the washed medium by tangential microfiltration.

8. The concentrate according to claim 1, wherein the adaptation of the bacteria is determined by measuring parameters of the bacteria culture medium and/or parameters of the bacteria.

9. The concentrate according to claim 8, wherein the parameters of the culture medium are the pH, the osmotic pressure and/or the temperature.

10. The concentrate according to claim 9, wherein the parameter of the culture medium is the pH and the adaptation step is performed by reducing the pH by natural acidification.

11. The concentrate according to claim 1, wherein the bacteria are adapted by a tangential microfiltration method.

12. The concentrate according to claim 8, wherein the parameter of the bacteria is the size thereof.

13. The concentrate according to claim 12, wherein the distribution of lengths of each bacterium of said concentrate are primarily between 0.1 and 10 micrometers.

14. The concentrate according to claim 1, wherein its pH is between 3 and 6.

15. The concentrate according to claim 1, wherein it is preserved at a temperature between −50° C. and 4 C after packaging.

16. The concentrate according to claim 15, wherein it is reheated to a temperature between 25 C and 45 C, before being used.

17. A food additive comprising the concentrate according to claim 1.

18. (canceled)

19. A flexible, hermetically sealed and sterile bag containing the concentrate according to claim 1.

20. A food product comprising the liquid concentrate of adapted and viable bacteria according to claim 1.

21. The food product to claim 20, wherein it is a dairy product or a drink.

22. A method for producing a the food product according to claim 20, comprising adding the liquid concentrate of adapted and viable bacteria to the food product at the end of the production line and before packaging of the food product.

23. The method according to claim 22, wherein the liquid concentrate of adapted and viable bacteria is added to the food product in the line by pumping.
